(19) United States
(12) Patent Application Publication
(10) Pub. No.: US 2017/0273837 A1
(43) Pub. Date: Sep. 28, 2017

(54) Wound Care Accessory Device

(71) Applicant: Gina Brueckner, Palm Harbor, FL (US)

(72) Inventor: Gina Brueckner, Palm Harbor, FL (US)

(21) Appl. No.: 15/080,238

(22) Filed: Mar. 24, 2016

(51) Int. Cl. A61F 15/00; A61F 13/00

(52) U.S. Cl. CPC A61F 15/008; A61F 13/00085; A61F 2013/00161; A61F 2013/00272

(57) ABSTRACT

A wound care accessory device providing pre-cut tape and an infection control barrier includes a sheet that comprises a first section and a second section. The sheet is substantially rectangularly shaped. A plurality of tape strips is disposed on and peelable from the second section of the sheet. The sheet is configured for placement proximate to a wound that requires care. The sheet provides a barrier to control infection. The first section is configured for placement of accessories used in caring for the wound. The tape strips are positioned on the second section of the sheet. The strips are configured for peeling by the user from the sheet to be used in caring for the wound.

### BACKGROUND OF THE DISCLOSURE

### Field of the Disclosure

The disclosure relates to wound care accessory devices and more particularly pertains to a new wound care accessory device providing pre-cut tape and an infection control barrier.

### SUMMARY OF THE DISCLOSURE

An embodiment of the disclosure meets the needs presented above by generally comprising a sheet that comprises a first section and a second section. The sheet is substantially rectangularly shaped. A plurality of tape strips is disposed on and peelable from the second section of the sheet. The sheet is configured for placement proximate to a wound that requires care. The sheet provides a barrier to control infection. The first section is configured for placement of accessories used in caring for the wound. The tape strips are positioned on the second section of the sheet. The strips are configured for peeling by the user from the sheet to be used in caring for the wound.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is an isometric perspective view of a wound care accessory device according to an embodiment of the disclosure.

FIG. 2 is a top view of an embodiment of the disclosure.

FIG. 3 is a bottom view of an embodiment of the disclosure.

FIG. 4 is a cross-sectional view of an embodiment of the disclosure.

### DESCRIPTION OF THE PREFERRED EMBODIMENT

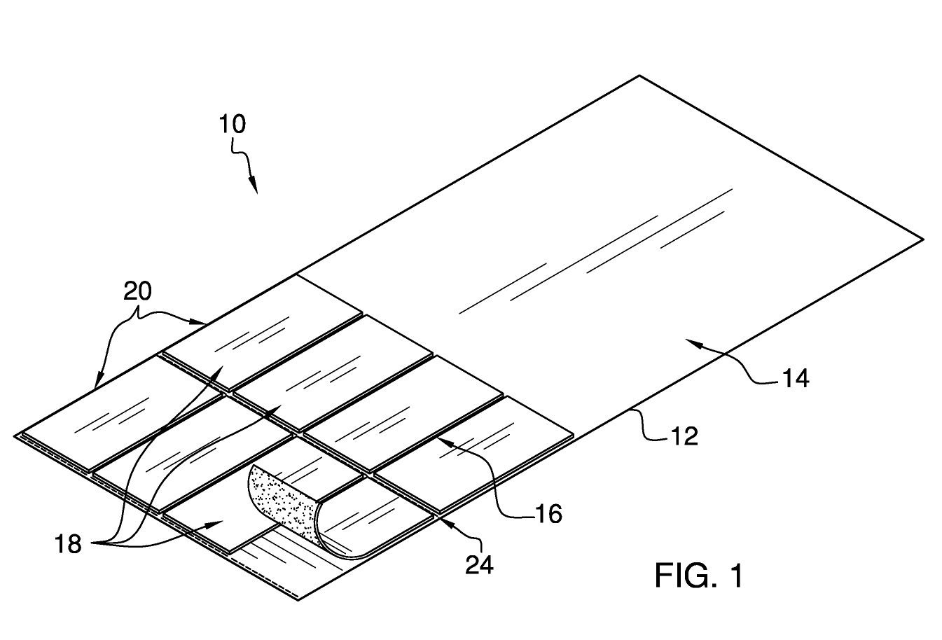

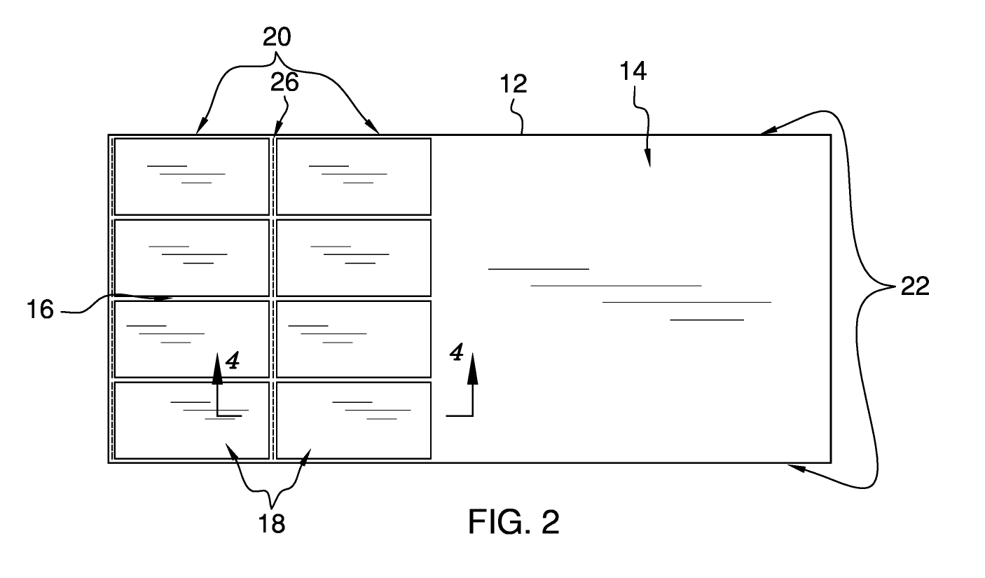

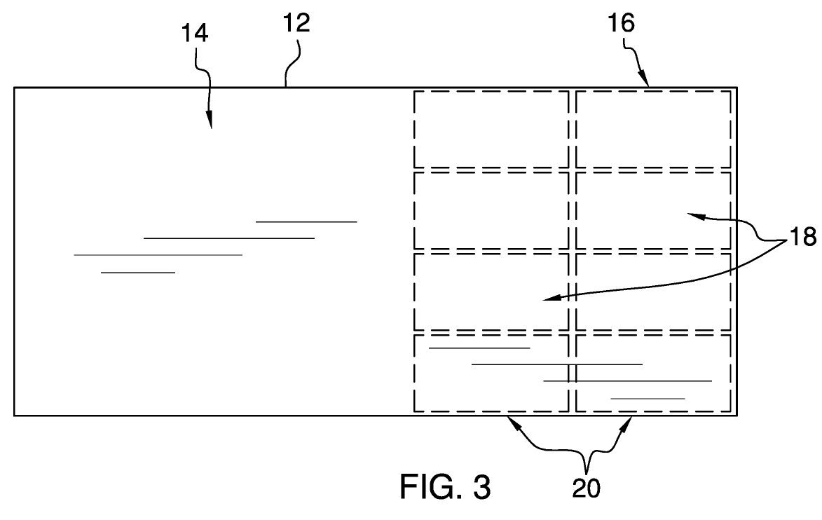

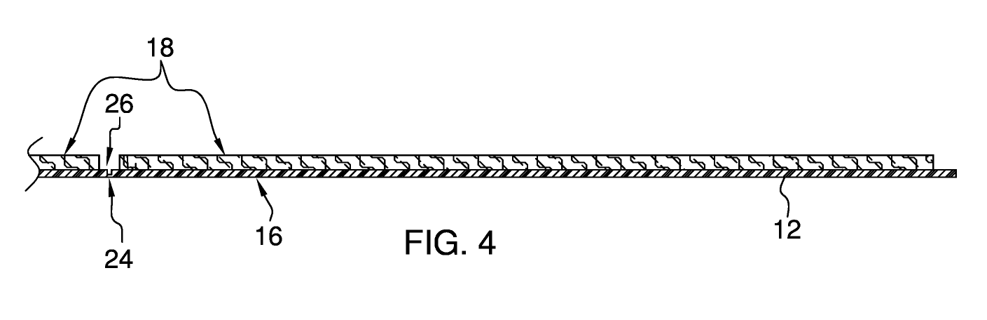

With reference now to the drawings, and in particular to FIGS. 1 through 4 thereof, a new wound care accessory device embodying the principles and concepts of an embodiment of the disclosure and generally designated by the reference numeral 10 will be described.

As best illustrated in FIGS. 1 through 4, the wound care accessory device 10 generally comprises a sheet 12 that comprises a first section 14 and a second section 16. The sheet 12 is substantially rectangularly shaped. The sheet 12 comprises paper. In another embodiment, the sheet 12 comprises wax-coated paper. A plurality of tape strips 18 is disposed on and peelable from the second section 16 of the sheet 12. The tape strips 18 are positioned in a plurality of rows 20 that extends between opposing side edges 22 of the sheet 12. The plurality of rows 20 comprises from one to four rows 20. In another embodiment, the plurality of rows 20 comprises two rows 20.

The plurality of tape strips 18 comprises from two to sixteen tape strips 18. In another embodiment, the plurality of tape strips 18 comprises from four to twelve tape strips 18. In yet another embodiment, the plurality of tape strips 18 comprises from six to ten tape strips 18. In yet another embodiment, the plurality of tape strips 18 comprises eight tape strips 18.

Each tape strip 18 is from four to twenty centimeters long and from one half to eight centimeters wide. In another embodiment, each tape strip 18 is from six to sixteen centimeters long and from two to seven centimeters wide. In yet another embodiment, each tape strip 18 is from eight to twelve centimeters long and from four to six centimeters wide. In yet another embodiment, each tape strip 18 is 10.16 centimeters long by 5.08 centimeters wide. The tape strips 18 comprise medical tape. The tape strips 18 comprise ostomy tape.

The present invention also anticipates the sheet 12 comprising a plurality of sheets that are reversibly coupled end-to-end. The plurality of sheets is configurable as a roll. An end sheet is grasped by the user and separated from the next sheet on the roll along a decoupling line defined by respective opposing ends of the end sheet and the next sheet on the roll. In one embodiment, the decoupling line comprises a plurality of penetrations.

Each of a plurality of separation lines 24 extends between the opposing side edges 22 of the sheet 12. Each separation line 24 is positioned between adjacent rows 20. The sheet 12 is separable at the separation line 24 such that the adjacent rows 20 are separated. The separation lines 24 comprise perforations 26 positioned through the sheet 12.

In use, the sheet 12 is configured for placement proximate to a wound requiring care. The sheet 12 provides a barrier to control infection. The first section 14 is configured for placement of accessories used in caring for the wound. The tape strips 18 are positioned on the second section 16 of the sheet 12. The tape strips 18 are configured for peeling by the user from the sheet 12 to be used in caring for the wound.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. Wound care accessory device comprising:
a sheet comprising a first section and a second section, said sheet being substantially rectangularly shaped;
a plurality of tape strips disposed on and peelable from said second section of said sheet; and
wherein said sheet is configured for placement proximate to a wound requiring care, wherein said sheet provides a barrier to control infection, such that said first section is configured for placement of accessories used in caring for the wound, and wherein said tape strips are positioned on said second section of said sheet such that said strips are configured for peeling by the user from said sheet to be used in caring for the wound.

2. The device of claim 1, further including said sheet comprising paper.

3. The device of claim 2, further including said sheet comprising wax-coated paper.

4. The device of claim 1, further including said strips being positioned in a plurality of rows extending between opposing side edges of said sheet.

5. The device of claim 4, further including said plurality of rows comprising from one to four said rows.

6. The device of claim 5, further including said plurality of rows comprising two said rows.

7. The device of claim 1, further including said plurality of strips comprising from two to sixteen strips.

8. The device of claim 7, further including said plurality of strips comprising from four to twelve strips.

9. The device of claim 8, further including said plurality of strips comprising from six to ten strips.

10. The device of claim 9, further including said plurality of strips comprising eight strips.

11. The device of claim 1, further including each said strip being from four to twenty centimeters long and from one half to eight centimeters wide.

12. The device of claim 11, further including each said strip being from six to sixteen centimeters long and from two to seven centimeters wide.

13. The device of claim 12, further including each said strip being from eight to twelve centimeters long and from four to six centimeters wide.

14. The device of claim 13, further including each said strip being 10.16 centimeters long by 5.08 centimeters wide.

15. The device of claim 1, further including said tape strips comprising medical tape.

16. The device of claim 1, further including said tape strips comprising ostomy tape.

17. The device of claim 4, further including a plurality of separation lines, each said separation line extending between said opposing side edges of said sheet, each said separation line being positioned between adjacent said rows, wherein said sheet is separable at said separation line such that said adjacent said rows are separated.

18. The device of claim 17, further including said separation lines comprising perforations positioned through said sheet.

19. Wound care accessory device comprising:
a sheet comprising a first section and a second section, said sheet being substantially rectangularly shaped, said sheet comprising paper, said sheet comprising wax-coated paper;
a plurality of tape strips disposed on and peelable from said second section of said sheet, said strips being positioned in a plurality of rows extending between opposing side edges of said sheet, said plurality of rows comprising from one to four said rows, said plurality of rows comprising two said rows, said plurality of strips comprising from two to sixteen strips, said plurality of strips comprising from four to twelve strips, said plurality of strips comprising from six to ten strips, said plurality of strips comprising eight strips, each said strip being from four to twenty centimeters long and from one half to eight centimeters wide, each said strip being from six to sixteen centimeters long and from two to seven centimeters wide, each said strip being from eight to twelve centimeters long and from four to six centimeters wide, each said strip being 10.16 centimeters long by 5.08 centimeters wide, said tape strips comprising medical tape, said tape strips comprising ostomy tape;
a plurality of separation lines, each said separation line extending between said opposing side edges of said sheet, each said separation line being positioned between adjacent said rows, wherein said sheet is separable at said separation line such that said adjacent said rows are separated, said separation lines comprising perforations positioned through said sheet; and
wherein said sheet is configured for placement proximate to a wound requiring care, wherein said sheet provides a barrier to control infection, such that said first section is configured for placement of accessories used in caring for the wound, and wherein said tape strips are positioned on said second section of said sheet such that said strips are configured for peeling by the user from said sheet to be used in caring for the wound.
